(19) United States
(12) Patent Application Publication
(10) Pub. No.: US 2025/0228587 A1
(43) Pub. Date: Jul. 17, 2025

(54) DEVICES, SYSTEMS, AND METHODS FOR PERFORMING THROMBECTOMY PROCEDURES

(71) Applicant: The Board of Trustees of the Leland Stanford Junior University, Stanford, CA (US)

(72) Inventors: Renee Zhao, Stanford, CA (US); Yilong Chang, Stanford, CA (US); Jeremy J. Heit, Los Altos, CA (US); Paul G. Yock, Atherton, CA (US)

(21) Appl. No.: 18/939,316

(22) Filed: Nov. 6, 2024

Related U.S. Application Data
(63) Continuation of application No. PCT/US23/21388, filed on May 8, 2023.
(60) Provisional application No. 63/339,504, filed on May 8, 2022.
(60) Provisional application No. 63/418,449, filed on Oct. 21, 2022.
(60) Provisional application No. 63/453,152, filed on Mar. 19, 2023.

(51) Int. Cl. A61B 17/3207

(52) U.S. Cl. CPC A61B 17/320758

(57) ABSTRACT

Thrombectomy devices are provided that include an elongated shaft including a proximal end coupled to a motor configured to spin the shaft, a distal end sized for introduction into a body lumen of a patient, and a longitudinal axis extending therebetween, the shaft configured to rotate about the axis, and a spinner tip on the distal end configured to generate localized suction adjacent the distal end when the shaft rotates. The spinner tip may be introduced into or provided within a lumen of a catheter including an outlet in a distal end thereof that is positioned adjacent a clot. With the spinner tip positioned adjacent the outlet, e.g., within the catheter lumen, the motor may be activated to generate localized suction, e.g., to dissolve the clot, reduce clot size, and/or prevent fragmentation of the clot.

### RELATED APPLICATION DATA

The present application is a continuation of co-pending International Application No. PCT/US2023/021388, filed May 8, 2023, which claims benefit of U.S. provisional applications Ser. No. 63/339,504, filed May 8, 2022, 63/418,449, filed Oct. 21, 2022, and 63/453,152, filed Mar. 19, 2023. The entire disclosures of Ser. Nos. 63/418,449 and 63/452,152 are expressly incorporated by reference herein.

### STATEMENT REGARDING FEDERALLY SPONSORED RESEARCH AND DEVELOPMENT

This invention was made with Government support under Contract 2145601 awarded by the National Science Foundation. The Government has certain rights in the invention.

### TECHNICAL FIELD

The present application relates to medical devices and, more particularly, to thrombectomy devices, and to systems and methods for performing thrombectomy or other medical procedures using such devices.

### BACKGROUND

Thromboembolism, usually referred as blood clot, is the result of blood coagulation in veins or arteries (see, e.g., FIG. 1A) that disrupts the normal flow of blood to a part of the body. Blood clots can occur at many locations in the body (e.g., as shown in FIG. 1B); for example, a clot located in a deep vein in a leg or arm may cause deep vein thrombosis (DVT), and a clot may travel from a deep vein to a lung to cause a pulmonary embolism (PE), or from an artery to the brain to cause a stroke, which are life-threatening conditions. Depending on the location within a subject's vasculature, thrombi can result in venous thromboembolism, pulmonary embolism, cerebrovascular stroke, peripheral artery occlusion, coronary thrombus, and/or acute myocardial infarction.

Based on the CDC, venous thromboembolism affects around 900,000 Americans each year. As many as 100,000 people die of blood clots each year. One of four people who have a PE die without warning. More sadly, PE is a leading cause of death in a woman during pregnancy or just after having a baby. Blood clots are also a leading cause of death in people with cancer after the cancer itself. It costs up to ten billion dollars each year in the US to address problems related to blood clots, and the treatment can be as much as $15,000 to $20,000 per person and often results in readmission to the hospital.

Acute Ischemic Stroke (AIS) is the leading cause of disability and the fifth leading cause of death in the United States. AIS results from blockage or interruption of blood flow within a cervical or cerebral artery, and this lack of blood flow to the brain may result in irreversible brain injury (core infarction) or impaired neuronal function in ischemic, but potentially salvageable, brain tissue (penumbra). AIS may be treated with intravenous thrombolysis within 3-4.5 hours of symptom onset, but fewer than five percent of AIS patients reach medical care within this time frame.

Percutaneous thrombectomy is a minimally invasive interventional treatment, during which the surgeon inserts a catheter into the patient's blood vessel to remove the blood clot and restore blood flow to the affected area. There are two commonly used mechanical thrombectomy technologies to remove large clots: (i) aspiration thrombectomy, where a continuous vacuum aspiration is induced through a guide catheter to suction out the clot; (ii) stent retriever thrombectomy, where a mesh tube is used pull out the clot, e.g., as shown in FIG. 2A. However, the use of stent retrievers and aspiration devices is always associated with a risk of thrombus fragmentation, e.g., as shown in FIG. 2B, during which the big clot may break into small pieces (100 μm˜1000 μm) and travel downstream in the blood vessel, potentially blocking the blood flow at multiple other locations and/or leading to new life-threatening emboli that may require emergent open surgeries.

More recently, endovascular thrombectomy using aspiration or a stent-retriever device, such as that shown in FIG. 2A, has been shown to be an effective treatment for AIS that involves large vessel occlusion (AIS-LVO) of the internal carotid, proximal middle cerebral, proximal anterior cerebral arteries, or basilar artery up to twenty-four hours from symptom onset. Thrombectomy has led to a marked improvement in AIS-LVO patient outcomes, and it has become the standard of care for AIS patients with an AIS-LVO.

Despite the recent advancements in AIS treatment, there are significant gaps in knowledge and available devices that limit optimal treatment of AIS treatments. Current thrombectomy techniques fail to restore any or insufficient blood flow in about fifteen percent (15%) of patients after multiple passes, with aspiration methods having a failure rate of about twenty-five to thirty three percent (25-33%). Common reasons for failed thrombectomy include clots with high fibrin content, clot fragmentation that prevents complete removal, and clots that are resistant to modern thrombectomy devices. In addition, recent data indicate that in order to be maximally effective, thrombectomy should restore about ninety-five to one hundred percent (95-100%) of the blood flow distal to the site of arterial blockage, and this blood flow should be restored within one thrombectomy attempt (“first pass effect”) to maximize the likelihood of achieving a good outcome. However, the first pass effect is achieved in fewer than about fifty percent (50%) of patients who undergo thrombectomy, and there is a substantial need to develop new thrombectomy devices to improve the thrombectomy process.

Therefore, improved devices and methods for performing thrombectomy procedures would be useful.

### SUMMARY

The present application is directed to medical devices and, more particularly to thrombectomy devices, and to systems and methods for performing thrombectomy or other medical procedures using such devices. In one example, the devices may include a spinner device introduced through a catheter or other tubular member (or directly into a body lumen) that mechanically reduces or compresses the volume and/or separates components of the clot (e.g., red blood cells, from fibrin or other residual fiber material) and/or dissolves or partially dissolves the clot, e.g., by coupled suction-induced compression and shear load applied to the clot by the spinner. That is, it should be understood that the terms “reducing”, “reduce”, or “reduces” with respect to a clot, include any manner in which a clot volume is reduced, including by separating components of the clot, e.g., red blood cells, from fibrin or other residual fiber material and/or compressing the clot and/or dissolving or partially dissolving the clot and/or reducing the volume of the residual clot material, e.g., to facilitate removal of the residual material. For example, the spinner may rotate rapidly to squeeze out red blood cells (RBCs) in a clot leaving a compacted fibrin fiber network, which may then be captured by the spinner tip, aspirated into the catheter, and/or otherwise removed from the vessel.

In addition or alternatively, suction may be applied to enhance reducing the clot and/or preventing fragmentation, suction generated by the spinner or another source applying a vacuum to the treatment site. Optionally, additional devices may be provided that may engage the clot to enhance spinning, e.g., one or more wires extending from the spinner or another device introduced into the treatment site separately. Optionally, a jet of saline or other fluid may also be directed into the treatment site to spin and/or otherwise enhancing dissolving the clot. Thus, the devices and systems herein may help to dissolve clots, reduce the size of blood clots, reduce thrombus fragmentation, and/or prevent large segmental debris from traveling downstream, which may reduce risks during interventional/endovascular procedures.

In accordance with one example, a thrombectomy device is provided that includes an elongated shaft comprising a proximal end configured to be coupled to a controller to spin the shaft, a distal end sized for introduction into a body lumen of a patient, and a longitudinal axis extending therebetween, the shaft configured to rotate about the axis; and a spinner member or element on the distal end configured to generate localized suction and/or shear force adjacent the distal end when the shaft rotates to reduce or dissolve a clot, reduce clot size, and/or prevent fragmentation of a clot within the body lumen. For example, the spinner tip may include an annular body extending distally from the distal end such that an opening communicating with a cavity within the annular body may be positioned adjacent the clot to apply the localized suction and/or shear force to the clot. Optionally, the spinner tip may include one or more blades or other external features on the annular body and/or one or more slits or other openings in the wall of the annular body, e.g., to enhance the localized suction that is generated at the opening and within the cavity.

In accordance with another example, a thrombectomy device is provided that includes a catheter or other tubular member including a proximal end, a distal end sized for introduction into a body lumen adjacent clot, and a lumen extending from the proximal end to an outlet in the distal end; an elongated shaft comprising a proximal end configured to be coupled to a controller to spin the shaft, a distal end sized for introduction into the lumen, and a longitudinal axis extending therebetween, the shaft configured to rotate about the axis; optionally, a sleeve surrounding the shaft; and a spinner tip on the shaft distal end configured to generate localized suction and/or shear force adjacent the outlet when the shaft rotates to reduce clot size of clot within a body lumen adjacent the outlet, e.g., by applying localized compression and/or shear forces to the clot to squeeze out red blood cells (RBCs) leaving a compacted fibrin fiber network. Optionally, the fibrin network or other residual material may then be removed, e.g., aspirated into the tubular member and/or captured by the spinner tip, which may be withdrawn to remove the residual material.

In accordance with still another example, a system is provided for performing a thrombectomy procedure that includes a tubular member including a proximal end, a distal end sized for introduction into a body lumen adjacent clot, and a lumen extending from the proximal end to an outlet in the distal end; an elongated shaft comprising a proximal end, a distal end sized for introduction into the lumen, and a longitudinal axis extending therebetween; a motor and/or controller coupled to the proximal end of the shaft to rotate the shaft about the axis; and a spinner tip on the shaft distal end configured to generate localized suction and/or shear force adjacent the outlet when the shaft rotates to reduce the size of clot within a body lumen adjacent the outlet.

In accordance with yet another example, a method is provided for performing thrombectomy that includes introducing a spinner device into a body lumen adjacent a target blood clot; and rotating the spinner device to generate localized suction to dissolve the clot, reduce clot size, and/or prevent fragmentation of the clot.

In accordance with another example, a tethered biomedical device is provided that includes an elongated tether having a proximal end and a distal end, and a tool head disposed on the distal end of the tether. The tether is an elongated, flexible member, such as a shaft, cable, tubular member, guidewire, or similar apparatus. The tether is configured to be advanced along a pathway within the body, including body lumens and/or body cavities and, optionally, one or more catheters, introducer sheaths, guidewires, or other delivery devices introduced into any such pathway. For example, the tether may be navigated by a combination of pushing, pulling, and rotating to steer the device to a target location, e.g., within a catheter or other delivery device. The tether is also configured to be rotated by a rotational driver to spin the tool head.

The tool head may be configured to perform ablation of body tissue and/or create suction, e.g., to reduce or dissolve clots. For example, the tool head may include a tubular body having a lumen extending axially along a central axis of the tubular body. Optionally, the tool head may include one or more blades disposed on, and extending radially outward from, the exterior surface of the tool head, which may enhance the suction and/or ablative function of the tool head.

In one example, the blades may be straight, raised ribs on the exterior surface of the tubular body and aligned parallel to the central axis of the tubular body.

In another example, the tubular body may include a plurality of holes through the wall of the tubular body. The holes in the tubular body may improve the localized suction capability of the tethered biomedical device. The holes may be slits, apertures, or other through holes in the tubular body, which may help facilitate clot removal. In the case that the device has blades, the holes may be between the blades. The holes are angularly spaced around tubular body. For instance, the tubular body may have 2 holes angularly spaced by 180° around the tubular body, or 4 holes angularly spaced by 90° around the tubular body.

In examples in which the tool head has both blades and holes, the holes may be positioned between the blades. For example, the tubular body may have two blades and two holes positioned between the adjacent blades, or the tubular body may have four blades and four holes spaced between the adjacent blades.

In accordance with still another example, a method is provided for using a tethered biomedical device. The tethered medical device is introduced into a patient's body via a small incision. Optionally, an introducer is inserted into the incision and the tool head is inserted into the body through the introducer. The tethered medical device is navigated to position the tool head proximate a target location within the body by pushing the tether and steering the tether through a pathway within the body, including body lumens and/or body cavities. Once the tool head is advanced to the target location, the tool head is used to perform a biomedical procedure, such as a diagnostic or treatment procedure. After performing the biomedical procedure, the tethered medical device is retracted from the body by retracting the tether, same or similar to advancing the device to the target location, except in the opposite direction.

In another aspect of the method, the biomedical procedure is an ablation procedure performed by spinning the tool head while bearing the tool against body tissue at the target location to remove the body tissue. The tool head may be translated, and its orientation adjusted, to position the tool head to ablate the body tissue by manipulating the tether. The tool head is spun by spinning the tether using the rotational driver. In still another aspect, the body tissue is an occlusion, such as thrombus and/or plaque, within a blood vessel.

In another aspect, the method may include using the tethered medical device to capture and remove an object (e.g., material such as a blood clot or tissue ablated by the tool head) at the target location. In this aspect, the tool head is positioned proximate the object and then the tool head is rotated by spinning the tether using the rotational driver. The spinning tool head creates a suction (a low-pressure zone) within the lumen of the tubular body which pressurizes and/or compresses the object toward a distal face of the tool head. The object may be sucked into the lumen of the tubular body, or pressed close to the tool head. The tool head may then be retracted along the pathway by pulling the tether, e.g., while spinning the tool head or with the tool head stationary, to pull the object along the pathway and out of the body.

Other aspects and features of the present invention will become apparent from consideration of the following description taken in conjunction with the accompanying drawings.

### BRIEF DESCRIPTION OF THE DRAWINGS

It is believed the present invention will be better understood from the following description of certain examples taken in conjunction with the accompanying drawings, in which like reference numerals identify the same elements and in which:

FIG. 1A shows an example of a blood clot in a blood vessel.

FIG. 1B is a schematic showing how blood clots at different locations in a body can cause deep vein thrombosis (leg/arm), pulmonary embolism (lung), and stoke (brain).

FIGS. 2A and 2B show an example of mechanical thrombectomy using a stent retriever, showing the thrombus fragmentation that may result during such a procedure.

FIG. 3 shows an example of a thrombectomy device including a catheter or sheath and a spinner connected to a flexible shaft insertable into the catheter.

FIG. 4 shows an exemplary spinner tip attached to a distal end of a shaft, which may be included in the device of FIG. 3.

FIGS. 4A-4C show exemplary spinner tips that may be included in the device of FIG. 3.

FIG. 4D shows the spinner tip of FIG. 4C being loaded with a drug delivery member.

FIGS. 5A and 5B are cross-sectional views of a blood vessel showing an exemplary method for dissolving a blood clot using the device of FIG. 3.

FIGS. 6A-6C show an example of clot size reduction that may be achieved using a thrombectomy device including a spinner, such as that shown in FIG. 3. In the example shown in FIG. 6A, the spinner successfully removed substantially all RBCs from the clot in about three minutes. The appearance of clot before and after spinning, as shown in FIGS. 6B and 6C, respectively, shows the size reduction to less than 10% of the initial volume.

FIG. 7 is an exemplary SEM image of a blood clot showing the clot is mainly composed of RBCs trapped in fibrin fiber networks.

FIGS. 8A and 8B show SEM images of a clot, e.g., the clot in FIGS. 6B and 6C, respectively, showing that the original clot contains >80 vol % of RBC and, after spinning, the white clot is a highly densified fibrin fiber network.

FIG. 9A is a graph showing an example of CFD results on pressure drop in a spinner under different spinning frequencies, demonstrating higher frequency spinning leads to larger pressure drop for better suction.

FIG. 9B is a graph showing examples of centerline pressure profile of a spinner device spinning at various frequencies.

FIG. 9C is a graph showing examples of maximum centerline pressure drop comparison at various blade lengths of a spinner device spinning at various frequencies.

FIGS. 10(1) to 10(10) show examples of CFD results on pressure distribution at the centerline of a milli-spinner (FIG. 10) with different blade sizes during spinning.

FIGS. 11A and 11B show exemplary images from a particle image velocimetry (PIV) system used to evaluate and optimize the suction performance of a milli-spinner. In this example, suction performance of a 2.5 mm spinner is shown with a spinning frequency is 1600 rpm. The arrows denote the fluid velocity field.

FIGS. 12A and 12B show an example of an advancer device that may be provided on a proximal end of a thrombectomy device, such the device shown in FIG. 3.

FIG. 13 is a graph showing experimental efficacy results of a spinner device, under a spinning speed of forty thousand rpm, to reduce 0.03 gram, 0.05 gram, 0.07 gram, and 0.09 gram of formed clots to 30% of initial volume within a tube with water flowing to represent blood flow within a vessel.

FIG. 14 is a graph showing exemplary release rates of a drug carried within a spinner tip depending on rotation speed of the spinner tip.

FIGS. 15A and 15B visually demonstrate exemplary drug release by a spinner tip during experiments at spinner speeds of forty thousand rpm (FIG. 15A) and ten thousand rpm (FIG. 15B) with the intensity of color representing the differences in release rate.

FIG. 16 is a graph comparing localized suction that may be generated by the spinner tips shown in FIGS. 4A-4C.

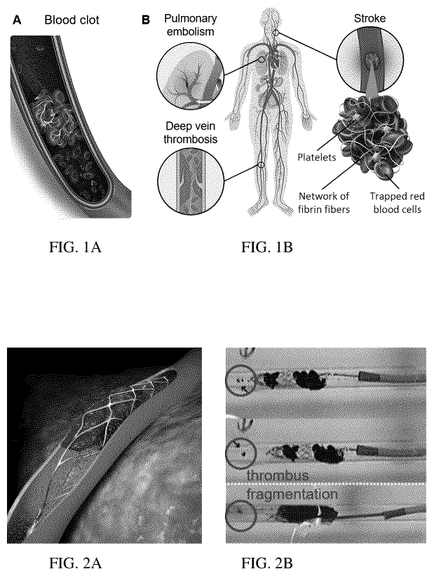

The drawings are not intended to be limiting in any way, and it is contemplated that various examples of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

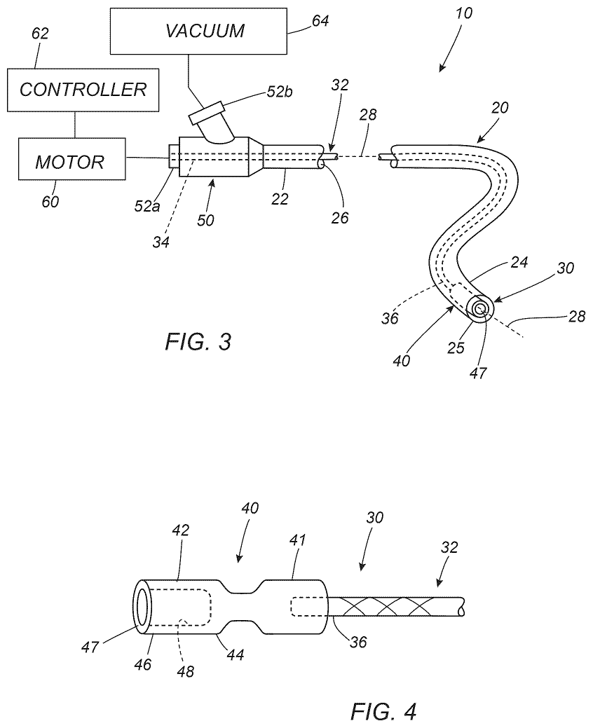

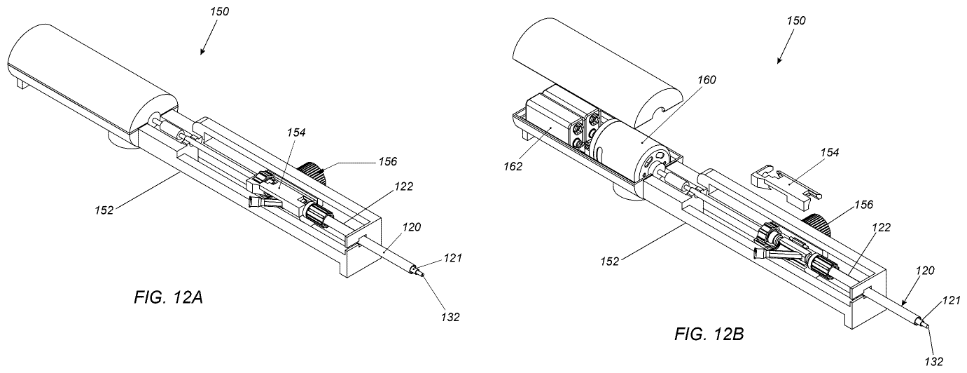

Turning to the drawings, FIG. 3 shows an example of a thrombectomy device or apparatus 10 that may be used with the systems and methods described herein throughout. In general, apparatus 10 includes an outer catheter, sheath, sleeve, or other member 20, and a spinner device 30 including a spinner tip or member 40 coupled to a shaft 32 that may be introduced through or otherwise positioned in the catheter 20. Optionally, the device 10 may include a sleeve (not shown in FIG. 3, see, e.g., sleeve 121 shown in FIGS. 12A and 12B), which may be positioned and/or otherwise provided around the shaft 32. The sleeve may help protect the inner surface of the catheter 20 when the shaft 32 rotates at high speeds, may reduce vibration resulting from the shaft 32 and/or spinner device 30 rotation, and/or may facilitate centering and/or stabilizing the spinner device 30.

Generally, as shown, the catheter 20 is an elongate tubular member including a proximal end 22 including a handle or hub 50, a distal end 24 sized for introduction into a blood vessel or other body lumen, and one or more lumens 26 extending between the proximal and distal ends 22, 24, e.g., along a longitudinal axis 28. For example, as shown, a main lumen 26 may be provided that communicates with one or more ports 52 in the handle 50 and extends to an outlet 25 in the distal end 24. Optionally, the catheter 20 may include one or more additional lumens extending at least partially between the proximal and distal ends 22, 24, e.g., a guidewire lumen for receiving a guidewire or other rail, a steering element lumen, and the like (not shown). It should also be understood that while catheter 20 is shown here as being tubular, it need not have a perfect circular cross-section. Indeed, it may be partially tubular, or have any other suitable geometry. Similarly, when reference is made to a lumen, it should be understood that a lumen may be a partial lumen, groove, or slit.

The catheter 20 may be constructed using conventional biocompatible materials and/or methods, e.g., formed from plastic, various polymers, metal, composite materials, having a substantially homogenous construction between the proximal and distal ends 22, 24. Alternatively, the construction may vary along the length of the catheter 20 to provide desired properties, e.g., to provide a proximal portion that is substantially rigid or semi-rigid, e.g., providing sufficient column strength to allow the distal end 24 of the catheter 20 to be pushed or otherwise manipulated from the proximal end 22, while a distal portion adjacent the distal end 24 may be substantially flexible to facilitate advancement through tortuous anatomy. In either construction, the catheter 20 may also be coated or layered (e.g., with a lubricious material) to aid in advancement.

The shaft 32 of the spinner device 30 may be an elongated flexible member including a proximal end 34 and a distal end 36 sized to be received within the lumen 26 of the catheter 20. The shaft 32 may be sufficiently long to allow introduction of the distal end 36 into a target blood vessel, e.g., through or along with the catheter 20, while the proximal end 34 remains outside the patient's body. For example, the shaft 32 may be a solid or tubular cable, e.g., including a plurality of helically wound inelastic fibers, wires, and the like, constructed to translate rotation from the proximal end 34 to the distal end 36 to rotate the spinner tip 40, e.g., with sufficient torsional strength such that rotation of the proximal end 34 causes directly corresponding rotation of the distal end 36, and consequently the spinner tip 40 at relatively high speeds, when coupled to a motor 60 and/or controller 62, as described further elsewhere herein as described elsewhere herein.

Optionally, a sleeve or other tubular member (not shown) may be provided around the shaft 32, e.g., to prevent the shaft 32 from contacting the inner wall of the catheter 20 when introduced into the lumen 26 and rotated by the motor. The sleeve may be formed from lubricious material, e.g., PTFE, and/or may include a coating on an inner surface thereof to reduce friction and/or otherwise facilitate the shaft 32 rotating in the sleeve. The sleeve may be axially fixed relative to the shaft 32, e.g., such that the sleeve extends from the proximal end 34 to the distal end 36 immediately proximal to the spinner tip 40. Alternatively, the sleeve may be separate from the spinner device 30, e.g., such that the sleeve may be introduced into the lumen 26 of the catheter 20 before introducing the spinner tip 40 and shaft 32.

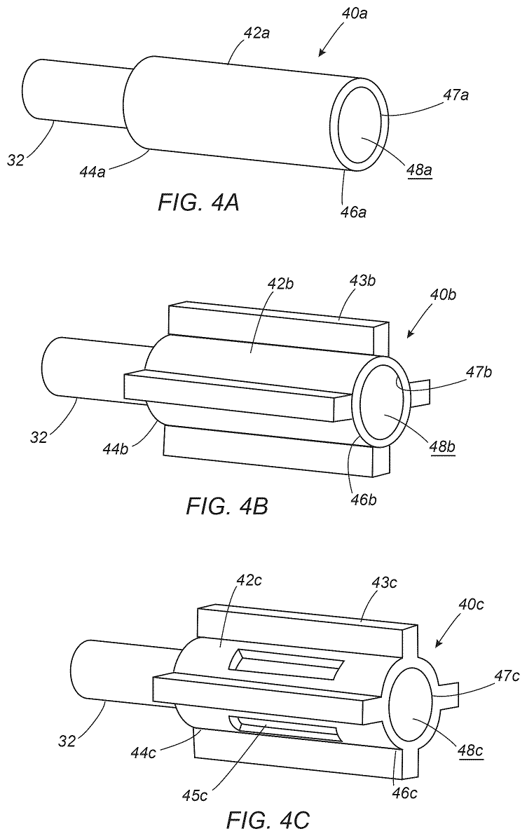

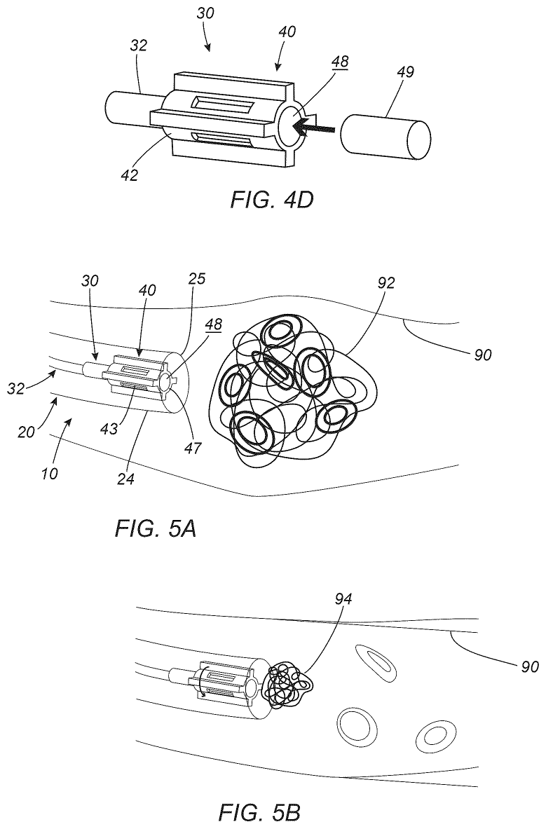

Generally, as shown in FIG. 4A, the spinner tip 40 may include a cylindrical or other annular body 42 including a closed proximal end 44 that may be connected to the shaft 32, and an open distal end 46 including an inlet 47 communicating with an interior cavity 48 of the body 42. Optionally, a hub 41 may be provided on the proximal end 44, which may facilitate attaching the spinner tip 40 to the distal end 36 of the shaft 32. For example, the hub 41 may be used to substantially permanently attached the tip 40 to the shaft 32, e.g., by one or more of over-molding, fusing, sonic welding, cooperating connectors, and the like. For example, as shown, the hub 41 may include a recess that may receive the distal end 36 of the shaft 32, and the hub 41 may be bonded, melted, press-fit, and/or otherwise permanently attached over the shaft 32.

Optionally, the spinner device 30 may include one or more markers, e.g., radiopaque rings or deposited material, at desired locations, e.g., on the distal end 36 of the shaft 32 and/or on the spinner tip 40, which may facilitate monitoring introduction and/or operation of the device 30 during a procedure, e.g., using fluoroscopy, Xray, ultrasound, or other external imaging. In addition or alternatively, the shaft 32 may be constructed from radiopaque materials, which may facilitate monitoring the shaft 32 during introduction and/or manipulation during a procedure.

Turning to FIGS. 4A-4C, examples of spinner tips 40 are shown that may be used with the devices, systems and methods provided herein, e.g., on the distal end 36 of the shaft 32. For example, in FIG. 4A, the spinner tip 40a includes a cylindrical body 42a that includes a substantially uniform annular wall extending between the proximal end 44a and the distal end 46a, which includes inlet 47a communicating with the interior cavity 48a. The body 42a may have a substantially flat, e.g., atraumatic distal face, which may facilitate placement against clot without macerating the clot when the body 42a is rotated. Optionally, as shown in FIG. 4B, the tip 40b may include one or more blades, struts, or other exterior features on the cylindrical body 42b to enhance localized suction and/or shear force on the clot, e.g., a plurality of elongate blades 43b extending at least partially between the proximal and distal ends 44b, 46b of the cylindrical body 42b. As shown, four blades 43b are shown that are spaced evenly around the cylindrical body 42b, e.g., offset about ninety degrees from one another around the circumference of the wall of the body 42b. Also as shown, the blades 43b may extend the length of the cylindrical body 42b or may only extend partially between the proximal and distal ends 44b, 46b. It should be understood that while equal spacing is shown here, unequal spacing is also contemplated. In addition, while four blades are shown, any number of blades may be used, e.g., two, three, four, five, six, seven, etc. Similarly, it should be understood that the blades need not be of equal dimension (e.g., length, width, height). In some variations, the blades may be of unequal dimensions.

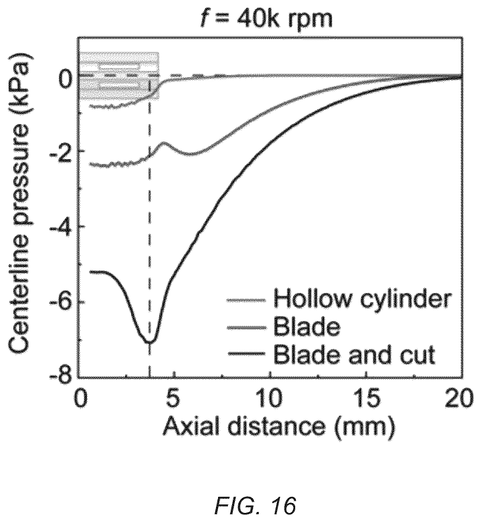

In addition or alternatively, as shown in FIG. 4C, the tip 40c may include one or more slits or other openings in the wall of the cylindrical body 42c, e.g., extending radially outwardly from the cavity 48c through the wall to the outer surface of the annular body 42c. For example, as shown, an elongate slit 45c is provided between each adjacent blade 43c, the slit 45c extending partially between the proximal and distal ends 44c, 46c of the cylindrical body 42c. The slits 45c may enhance the localized suction within the cavity 48c and inlet 47c, as described elsewhere herein. Alternatively, slits may be provided only between some of the blades and/or a plurality of slits may be provided in a cylindrical body without blades (not shown), as desired. Although four slits and blades are shown in these examples, it will be appreciated that any desired number of slits and/or blades may be provided on the spinner tip spaced around the annular body, e.g., two, three, four, or more. Similarly, it should be appreciated that the slits need not be of equal size. Adding blades and adding both blades and slits to the spinner tip may substantially increase the localized suction that is generated by the spinner tips 40a-40c shown in FIGS. 4A-4C. For example, FIG. 16 shows experimental results comparing localized suction generated at 40,000 rom for spinner tips without blades (hollow cylinder only, similar to 40a) with spinner tips including blades (similar to 40b) and including both blades and slits (similar to 40c).

The various dimensions of the spinner tip 40 may be sized to be inserted into a target blood vessel and/or to generate desired localized suction pressures, as described further elsewhere herein. In some examples, the body 42 may have a length between about one to five millimeters (1.0-5.0 mm), e.g., about 4.2 mm, and a wall thickness between about 0.05-0.15 mm, e.g., about 0.09 mm or 0.15 mm; the inlet 47 may have a diameter between about 0.5-1.2 mm, e.g., about 0.72 mm or 1.2 mm. The blades 43 may have heights between about 0.1-0.7 mm, e.g., about 0.31 mm or 0.51 mm, and widths between about 0.1-0.5 mm, e.g., about 0.24 mm or 0.40 mm. The slits 45 may have lengths between about 0.5-2.5 mm, e.g., about 2.1 mm, and widths between about 0.2-0.8 mm around the radius, e.g., about 0.45 mm or 0.75 mm.

In another alternative, the spinner tip may include a plurality of helical blades (not shown), e.g., extending at least partially around the circumference of the annular body and/or along the length of the annular body. Optionally, in this alternative, one or more helical slits or other openings may be provided between one or more of the helical blades. It will be appreciated that one or more additional features may be provided on the outer surface of the annular body, in addition to or instead of the blades and/or slits, that may enhance the localized suction generated by the spinner tip.

Returning to FIG. 4, the spinner tip 40 (or any other examples described herein) may be integrally formed as a single piece, e.g., from plastic, metal, composite material, and the like, e.g., manufactured using one or more of 3D-printing, by molding, micro-injection molding, casting, machining, and the like. Alternatively, the annular body 42 and/or hub 41 may be formed from one or more substantially continuous processes, such as extrusion and the like, e.g., to allow manufacturing a single assembly that may be separated into individual tips. Optionally, the annular body may be formed without the slits and/or blades, which may be added or formed during subsequent processing of the annular body, if desired.

The spinner tip 40 may be substantially rigid or, alternatively, the material of the spinner tip 40 may be flexible or semi-rigid, e.g., formed from relatively soft material, such as elastomeric material, e.g., silicone, or soft plastics, such that the distal end 46 of the annular body 42 provides a substantially atraumatic tip that may minimize risk of damaging tissue contacted by the tip. For example, the spinner tip 40 may be formed from relatively soft material, such as an elastomeric material with stiffness less than 40 MPa. Thus, the spinner tip 40 may be able to recover from deformation and retain its shape, e.g., by bending less than about one hundred eighty degrees (180°) degrees and/or twisting less than about five hundred forty degrees) (540°).

Similarly, the blades 43 may be formed from flexible and/or soft material and/or may include rounded or other atraumatic outer edges, e.g., to prevent damaging the vessel wall and/or causing the clot to be macerated when the spinner tip 40 is rotated.

The spinner tip 40 may have a diameter or other outer cross-section sized to be received within the lumen 26 of the catheter 20 while allowing the tip 40 to rotate freely, e.g., providing clearance around the tip 40 within the lumen. For example, the tip 40 may have an outer diameter between about 1.2-2.5 millimeters, e.g., having an outer diameter of about two millimeters (2 mm) or less.

Optionally, as shown in FIG. 4D, the spinner tip 40 may include a drug delivery member 49, e.g., for delivering one or more therapeutic and/or diagnostic agents. For example, the drug delivery member 49 may include a cylindrical body sized to be received in the cavity 48 of the spinner tip 40. In one example, the drug delivery member 49 may be formed from porous material, e.g., such that one or more agents may be loaded into the porous material and released, e.g., when the spinner tip 40 is rotated within a blood vessel. In addition or alternatively, the drug delivery member 49 may be formed from bioabsorbable or dissolvable material, e.g., to release the one or more agents as the material dissolves or otherwise breaks down.

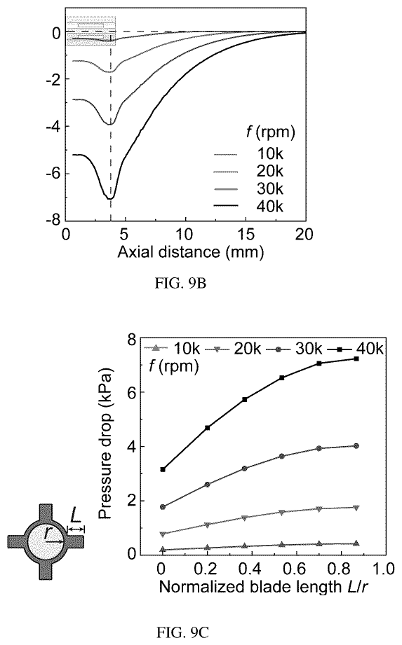

Returning to FIG. 3, the spinner tip 40 is connected to the shaft 32, which is coupled, in turn, to a motor 60 that provides the torque to rotate the spinner tip 40. For example, the proximal end 34 of the shaft 32 may include a connector (not shown) to couple the shaft 32 to the motor 60, e.g., via an external drive shaft, cable, and the like (also not shown). The motor 60 may be configured to rotate the spinner tip 40 at desired speeds, e.g., at least 1000 rpm, or at least 10,000 rpm, e.g., between about 1,000 and 200,000 rpm, between about 4,000 and 50,000 rpm, between about 10,000 and 40,000 rpm, between about 20,000 and 40,000 rpm, or between about 30,000 and 40,000 rpm, e.g., around 10,000 rpm or around 40,000 rpm, which may generate a desired localized suction pressure at the inlet 47 of the tip 40. For example, with the spinner tip rotating at 40,000 rpm, localized suction of about seven thousand Pascals (7 kPa) may be generated, while at 10,000 rpm, five hundred Pascals (0.5 kPa) may be generated, e.g., as shown in FIGS. 9A-9C.

In one example, the motor 60 may be configured to rotate the spinner tip 40 at a single set speed. Alternatively, the speed of the motor 60 may be variable, e.g., manually using an actuator of the controller 62 coupled to the motor 60, which may be adjusted by the user to modify the rotation speed of the spinner tip 40. Alternatively, the controller 62 may be configured to initially operate the motor 60 at a relatively lower speed and then the speed may be automatically increased to rotate the spinner tip 40 at a desired active speed. For example, the initial speed may be used to mechanically engage the clot, and then the speed may be increased, e.g., to rapidly remove red blood cells from the clot and reduce clot size, as described elsewhere herein.

Different from existing aspiration thrombectomy devices, which require continuous extraction of blood from the vessel being treated, the spinner devices herein may generate a highly localized suction force without removing any fluid from the vessel. Optionally, the devices may be used in conjunction with aspiration, e.g., by connecting a source of vacuum 64 to the catheter 20 or introducing a separate suction device (not shown), as described further elsewhere herein.

A controller 62 may be coupled to the motor 60 to control operation of the device 10, e.g., to allow a user to turn the motor 60 off and on to rotate the spinner tip 40 and generate the localized suction with a blood vessel. Optionally, the controller 62 may include one or more actuators, e.g., switches and the like (not shown), to activate/deactivate the motor and/or to adjust the speed, if desired. In addition or alternatively, the controller 62 may include an actuator (also not shown) to advance and/or retract the shaft 32 axially, e.g., relative to the catheter 20, e.g., using an advancer device 150, such as that shown in FIGS. 12A and 12B and described elsewhere herein. In another option, the controller 62 may include a robotic control system to control axial movement of the shaft 32 remotely, if desired.

In one example, the spinner device 30 and catheter 20 are assembled together, e.g., such that the catheter 20 and spinner device 30 are introduced together into a patient's body, e.g., similar to the device 150 shown in FIGS. 12A and 12B. In this way, the devices described herein are part of a pre-assembled system or kit. Alternatively, the spinner device 30 may be separate from the catheter 20, i.e., including the shaft 32 and spinner tip 40, e.g., such that the catheter 20 may be introduced initially into a patient's body, and, once the distal end 24 and outlet 25 are positioned adjacent a target clot, the spinner device 30 may be introduced into the catheter 20 and advanced to position the spinner tip 40 adjacent the outlet 25. In this way, the devices described herein may be assembled just prior to, or during, use. In this example, the handle 50 of the catheter 20 may include a port 52a that allows the spinner device 30 to be inserted into and removed from the lumen 26 of the catheter 20, which may include one or more hemostatic seals, e.g., to prevent fluid from leaking from the port 52a while allowing the shaft 32 of the spinner device 30 to be advanced through the port 52a into the lumen 26. Alternatively, the spinner device 30 may be permanently integrated with the catheter 20, e.g., such that the spinner device 30 cannot be removed but may be advanced and/or retracted axially within the lumen 26, e.g., using the advancer device 150.

Optionally, the catheter 20 and/or shaft 32 may include one or more stops or other safety features (not shown) to limit axial movement of the shaft 32 and thus help prevent accidental shearing of non-clot tissue during use. For example, a stop may be provided within the handle 50 that prevents the spinner device 30 from being advanced to expose the spinner tip 40 from the outlet 25 of the catheter 20. Alternatively, the stop(s) may allow the spinner tip 40 to be exposed partially or entirely from the outlet 25, if desired. Optionally, another stop may be provided that allows the spinner tip 40 to be retracted a desired distance proximally from the outlet 25, e.g., to allow residual fiber from a reduced or dissolved clot to be aspirated or otherwise directed into the outlet 25, as described further elsewhere herein. In another alternative, the spinner device 30 may be axially fixed relative to the catheter 20, e.g., such that the spinner tip 40 is located within the lumen 26 with the distal end 46 of the spinner tip 40 located immediately adjacent the outlet 25.

If the spinner device 30 is permanently integrated with the catheter 20, the proximal end 34 of the shaft 32 may extend from the port 52a on the handle 50 (whether the shaft 32 is movable axially or axially fixed). The proximal end 34 of the shaft 32 may include a connector configured to couple the shaft 32 to a driveshaft of the motor 60 (not shown), e.g., to allow the thrombectomy device 10 to be connected and disconnected from the motor 60. In this example, the thrombectomy device 10 may be a single-use, integral device that may be provided to a user for use during a procedure, after which the device 10 may be discarded. Alternatively, if the spinner device 30 is provided separately from the catheter 20, both may be single-use and/or disposable or one or both may be reusable, e.g., after cleaning and/or sterilization. In a further alternative, the spinner device 30 may be provided and/or introduced into a patient's body without the catheter 20, if desired.

Optionally, as shown in FIGS. 12A and 12B, an advancer device 150 may be provided on the proximal end of a thrombectomy device, e.g., in place of the handle 50 shown on the device 10 of FIG. 3. Generally, the advancer device 150 includes a stationary handle portion 152 coupled to a proximal end 122 of a catheter 120, and a slider portion 154 coupled to a shaft 132 carrying a spinner tip on its distal end (not shown). The catheter 120, shaft 132, and spinner tip may be generally constructed similar to any of the other devices herein, similar to the device 10 shown in FIG. 3. Optionally, a sleeve 121 may be provided around the shaft 132 that extends through the catheter 120 to protect the inner surface of the catheter 120 when the shaft 132 rotates, similar to other devices herein.

The components of the advancer device 150 may be provided within an outer housing, e.g., including clamshells or other portions that may be attached together (not shown), e.g., to protect the internal components. Optionally, the outer surface of the housing may be contoured to provide a grip to facilitate holding and/or manipulating the device, or the housing may include a base or other structure for stabilizing the housing relative to a patient during a procedure. Optionally, as shown in FIG. 12B, the advancer device 150 may include a motor 160 and/or a battery 162 or other power source, e.g., for driving the shaft 132 to rotate the spinner tip, similar to other devices herein.

As shown, the slider 154 may be slidably received in a track or other guide in the stationary portion 152, e.g., such that the slider 154 may be directed axially between a first or proximal position and a second or distal position, e.g., to advance and retract the spinner tip relative to the distal end of the catheter 120 (not shown), similar to other devices herein. As shown, a screw 156 or other actuator may be coupled to the slider 154, e.g., to allow an operator to manually direct the slider 154 between the first and second positions. Optionally, the screw 156 may include a fastener that may actuated to secure the slider 154 at a desired position, e.g., to fix the spinner tip relative to the distal end of the catheter 120, e.g., once deployed during a procedure. While a slider is shown here, any suitable actuation member and/or methods may be used, e.g., button, knob, and/or combinations thereof.

During use, with the spinner tip retracted within the catheter 120, the distal end of the catheter 120 may be introduced into a patient's body and advanced to a target location, e.g., adjacent a clot within the patient's vasculature (not shown). Once positioned, the screw 156 may be actuated to advance the spinner tip relative to the catheter 120, e.g., to place the distal face of the spinner tip against or immediately adjacent the clot, similar to other devices herein. The screw 156 may be used to lock the spinner tip's relative position in the catheter 120 once advanced to a desired location. The motor 160 may then be activated to rotate the spinner tip to dissolve and/or reduce the clot, similar to other devices herein. Once the clot is treated, the screw 156 may be actuated to retract the spinner tip back into the catheter 120, and the device may be removed (or directed to one or more additional locations to treat additional clot).

Any of the devices, systems, and methods described herein may optionally include a vacuum source. For example, as shown in FIG. 3, the device 10 may include a source of vacuum 64, e.g., a syringe, suction line, and the like (not shown), that may be coupled to the proximal end 22 of the catheter, e.g., to port 52b on the handle 50 for aspirating material into the lumen. For example, the port 52b may include a Luer fitting or other connectors that allow tubing from the source of vacuum 64 to be removably connected to the port 52b. Before or during advancing and/or activating the spinner tip 40 to reduce or dissolve a clot, the vacuum source 64 may be activated (or may be activated immediately upon advancing the spinner tip 40, if desired) to aspirate dissolved fibrin or other remaining clot material, e.g., into the lumen 26 of the catheter 20, as described further elsewhere herein. The spinner tip 40 may also help reorient and/or reposition the clot relative to the catheter 20, e.g., to enhance contact between the clot and the outlet 25, which may enhance vacuum suction from the lumen 26.

In addition or alternatively, a source of fluid, e.g., a syringe of saline, contrast, and the like, may be connected to the port 52b (or to a separate dedicated port, not shown, if desired). Thus, during use of the catheter 20, the lumen 26 may be flushed and/or the fluid may be delivered through the outlet 25, if desired.

Optionally, the thrombectomy device 10 and/or spinner device 30 may be included in a system or kit including one or more additional devices for use during a thrombectomy procedure. For example, the system may include an occlusion device and/or a capture member (not shown) to prevent fragments of a clot being treated from migrating elsewhere within the patient's vasculature. For example, such devices may be introduced and deployed from the catheter 20, e.g., through the lumen 26 or a secondary lumen. Alternatively, such devices may be introduced and deployed independently of the spinner device, e.g., via a separate catheter, sheath, or other device (not shown) downstream from the target clot.

Suitable additional devices for use within a system or kit as described herein may include, for example, a device carrying a balloon or other expandable member may be provided that may be introduced into a body lumen spaced apart from and/or adjacent the spinner tip 40 and/or catheter outlet 25, and the expandable member may be expanded to at least partially occlude the body lumen to prevent material from the clot migrating from the body lumen. Alternatively, a capture member may be provided for introduction into a body lumen adjacent the spinner tip, e.g., to capture residual fibrin material from the clot being treated by the spinner tip. Such a capture member may include a filter, a snare, a cage, and the like.

The devices and systems herein may be used during a thrombectomy procedure. For example, the spinner device 30 may be introduced into a body lumen, e.g., a vein, artery, and the like, adjacent a target blood clot, and the spinner tip 40 may be rotated to generate localized suction, e.g., to generate localized hydrodynamic forces combining compression and shear forces, to dissolve the clot, reduce clot size, and/or prevent fragmentation of a clot within the body lumen. For example, the spinner device may be deployed to reduce or otherwise dissolve a clot, e.g., by separating the red blood cells from the fibrin and/or other fibrous material. The red blood cells may simply be released within the vessel, e.g., such that the cells are metabolized by the body, and the fibrin may be captured, e.g., directly by the cavity of the spinner device, or using aspiration, a capture device, and the like, and/or treated with a thrombolytic drug or other agent to breakdown, dissolve, and/or otherwise neutralize the residual material before the spinner device is removed from the patient's vasculature.

In an exemplary method, shown in FIGS. 5A and 5B, the distal end 24 of the catheter 20 may be initially introduced into the patient's body, e.g., over a guidewire or other rail (not shown) positioned within the patient's vasculature from a percutaneous access site. The distal end 24 may be advanced to position the outlet 25 within a blood vessel 90 adjacent a target clot 92, as shown in FIG. 5A.

The spinner tip 40 may be introduced into the lumen 26 from the proximal end of the catheter 20 and advanced to position the inlet 47 adjacent the outlet 25 of the catheter 20 and, consequently, adjacent the clot 92, as shown in FIG. 5A. Alternatively, as described elsewhere herein, the spinner device 30 may be provided within the catheter 20, e.g., such that the spinner tip 40 is positioned adjacent the clot 92 at the same time as the catheter 20 is introduced. Optionally, a sleeve (not shown) may be provided around the shaft 32 within the lumen 26, which may be introduced with the spinner device 30 or may be introduced into the lumen 26 before inserting the spinner device 30.

Optionally, manipulation of the catheter 20 and/or spinner device 30 may be monitored using external imaging, e.g., fluoroscopy, Xray, ultrasound, and the like. For example, as described elsewhere herein, the spinner device 30 may include one or more radiopaque markers, e.g., on the distal end 36 of the shaft 32 and/or spinner tip 40, and/or the shaft 32 may be formed from radiopaque material that may be monitored to facilitate positioning the spinner tip 40 adjacent the clot 92. Optionally, contrast may be introduced into the blood vessel 90, e.g., via the port 52b, a separate port on the proximal end 22 of the catheter 20, or a separate device (not shown), to facilitate locating the clot 92 and positioning the spinner tip 40, which may be introduced through the catheter 20 (e.g., through the main lumen 26 or a separate lumen) or through a separate device. In addition or alternatively, the catheter 20 and/or spinner device 30 may be monitored using intravascular imaging systems and methods, such as intravascular ultrasound imaging, optical coherence tomography, and the like.

Once the device is positioned as desired relative to the clot 92, the spinner tip 40 may then be rotated, e.g., by activating the motor 60 (shown in FIG. 3), to generate localized suction within the inlet 47, thereby drawing the clot 92 against the distal end 24 of the catheter 20, e.g., to press the clot 92 against the distal end 46 of the spinner tip 40. As described above, the speed of the spinner tip 40 may be fixed or adjustable, e.g., manually by the user and/or automatically by the controller 60. For example, the spinner tip 40 may positioned against or immediately adjacent the clot 92 and rotated to generate shear forces that separate red blood cells from the clot. In one method, only the distal face of the spinner tip 40 is placed in contact with the clot and the spinner tip 40 is not rotated inside the clot 92, e.g., to prevent maceration.

Unlike other thrombectomy devices that attempt to catch or macerate the entire clot (leading to fragmentation of the clot into pieces), the devices and systems of the present disclosure help separate the red blood cells of the clot from the complex fibrin network, thus leading to greater efficiency and efficacy. That is, the spinning motion of the spinner tip 40 may generate a shear force which, in combination with the compression from the suction to squeeze out red blood cells trapped in the clot 92, which may escape through the slits 43 and/or otherwise from the cavity 48 of the spinner tip 40 into the vessel. Thus, unlike macerator devices, which mechanical break the clot into pieces and risk fragments traveling to other locations in the patient's body, the devices herein allow the red blood cells to be removed to reduce the volume of the clot without breaking up the residual fibrin material. The residual material may remain substantially intact and then removed, as described elsewhere herein. Further, macerator devices may be incapable of breaking up rich and/stiff fibrin networks of some clots and so may be incapable of removing such clots, while the devices herein allow such residual fibrin network to be captured and/or otherwise removed regardless of the stiffness of the residual material.

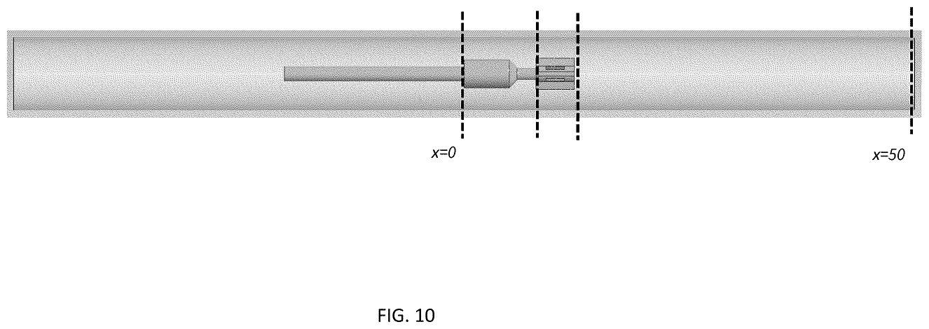

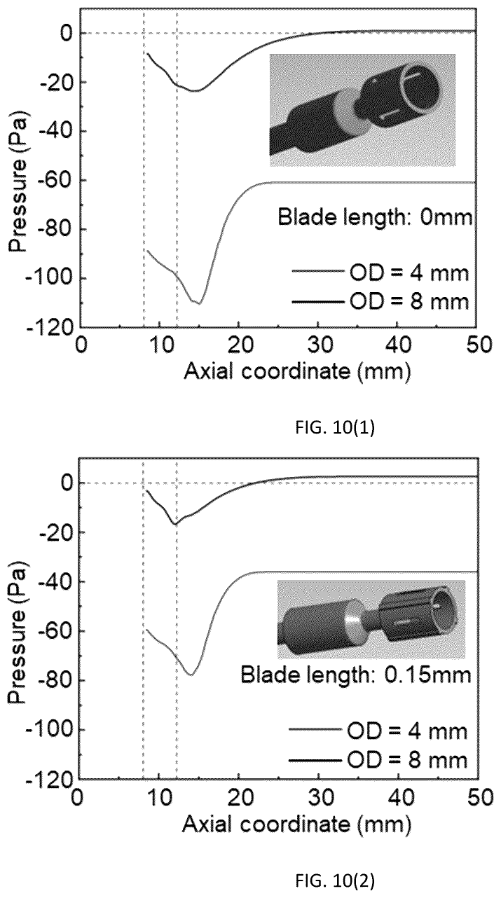

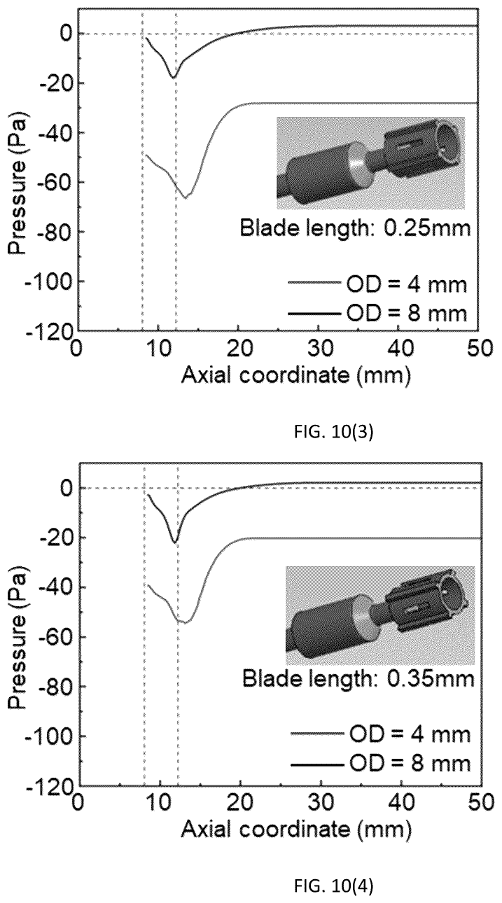

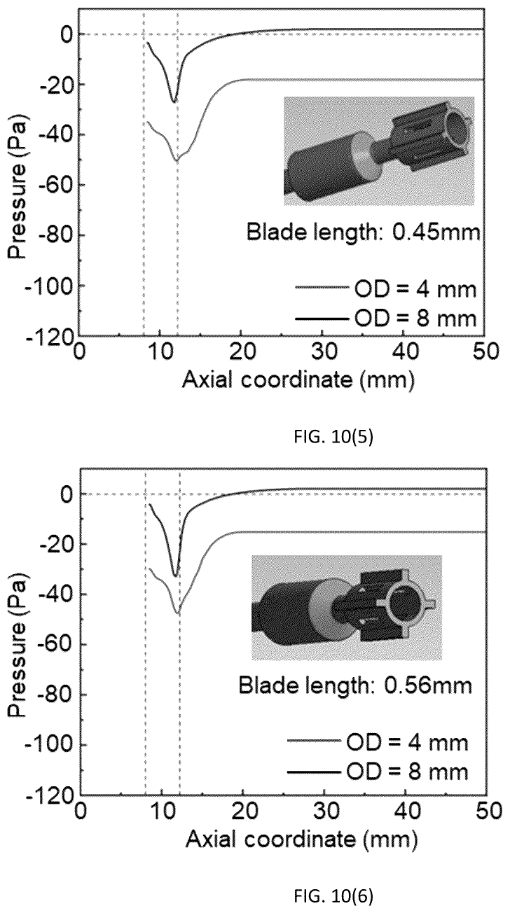

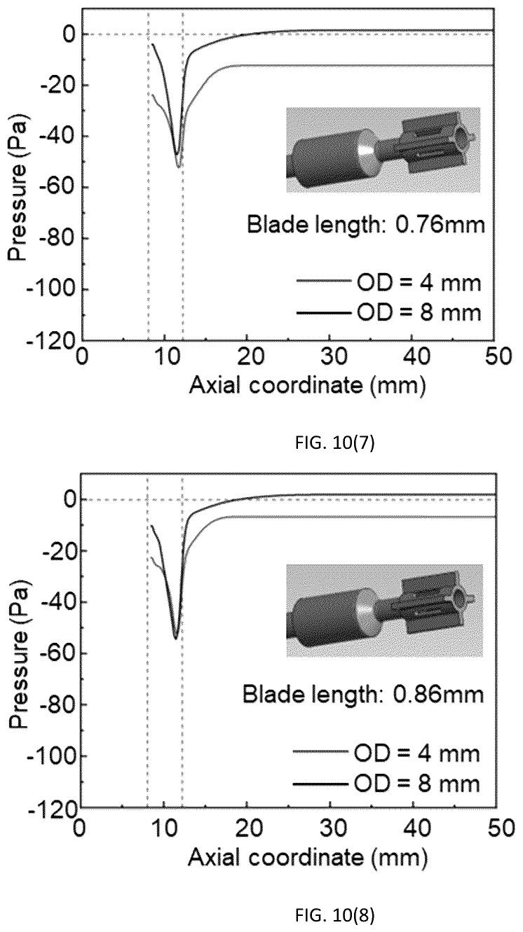

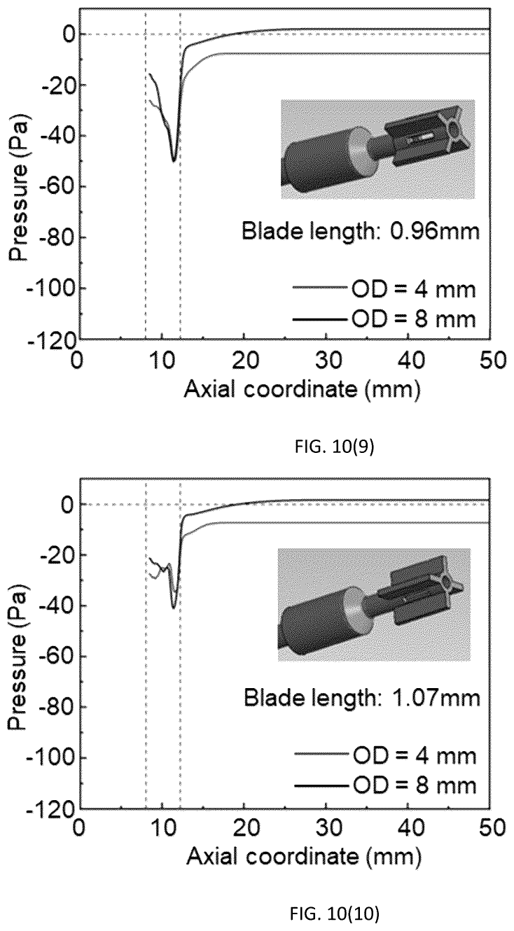

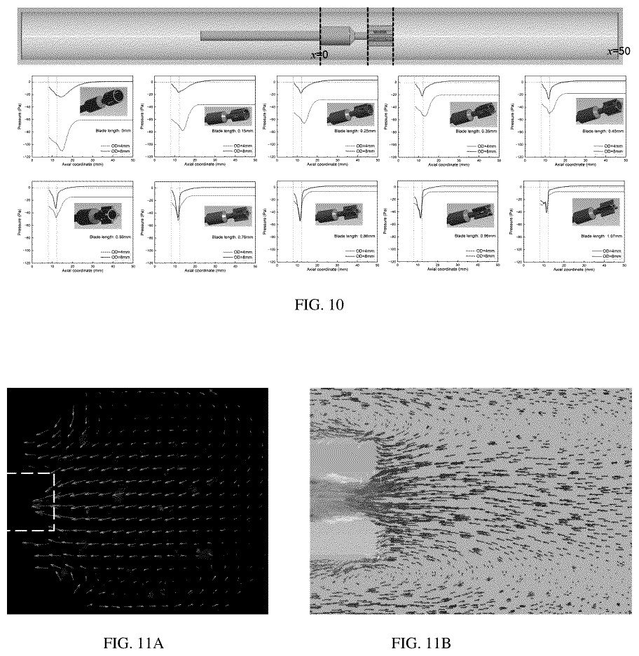

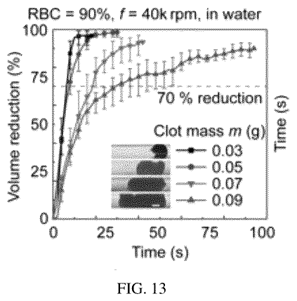

For example, as shown in FIGS. 9A-9C, increased spinning speed may lead to higher localized suction. The magnitude of suction is positively related to clot-dissolving efficacy as shown in FIG. 13. As shown in FIG. 13, the reduction effect is expressed as the time it takes for a clot to reach certain volume reduction amount (in this experiment, 70% volume reduction was the chosen as the evaluation standard). At 40,000 rpm, no breakup of the thrombus has been observed. At even higher spinning speeds, clot-dissolving efficiency is expected to increase. FIGS. 11A and 11B show exemplary pressures and localized flow that may be generated when the spinner tip 40 is rotated at various speeds, e.g., such as those shown in FIGS. 9A-9C and 10.

In this way, the spinner tip 40 may rapidly separate the red blood cells, e.g., in less than two minutes, leaving a compacted fibrin fiber network 94, e.g., as shown in FIG. 5B. Optionally, additional vacuum may be applied, e.g., by connecting a source of vacuum 64 to the proximal end 22 of the catheter 20, as shown in FIG. 3, to generate substantially continuous suction through the outlet 25 into the lumen 26. For example, when the spinner tip 40 is rotated while aspiration is also applied, the clot may be reduced in as little as five seconds or less. If desired, the spinner tip 40 may be rotated to mechanically break down the residual fiber network 94, which may be aspirated into the lumen 26 by the vacuum. In addition or alternatively, the spinner tip 40 may be retracted to draw the residual fiber network 94 into the lumen 26, e.g., using one or both of the localized suction generated by the spinner tip 40 and vacuum within the lumen 26. In this alternative, the catheter 20 may be removed from the blood vessel 90 once the residual fiber network 94 is captured, e.g., withdrawn entirely from the patient's body. As described above, the methods described herein throughout may also utilize devices having one or more stops for safety.

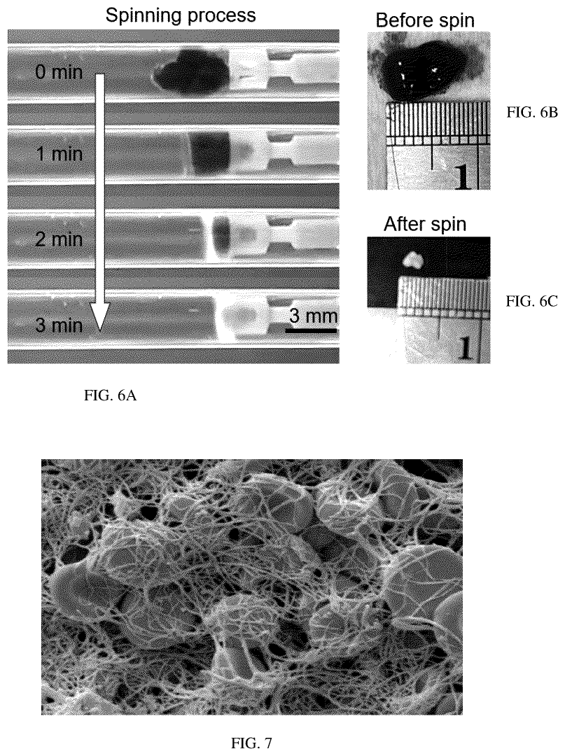

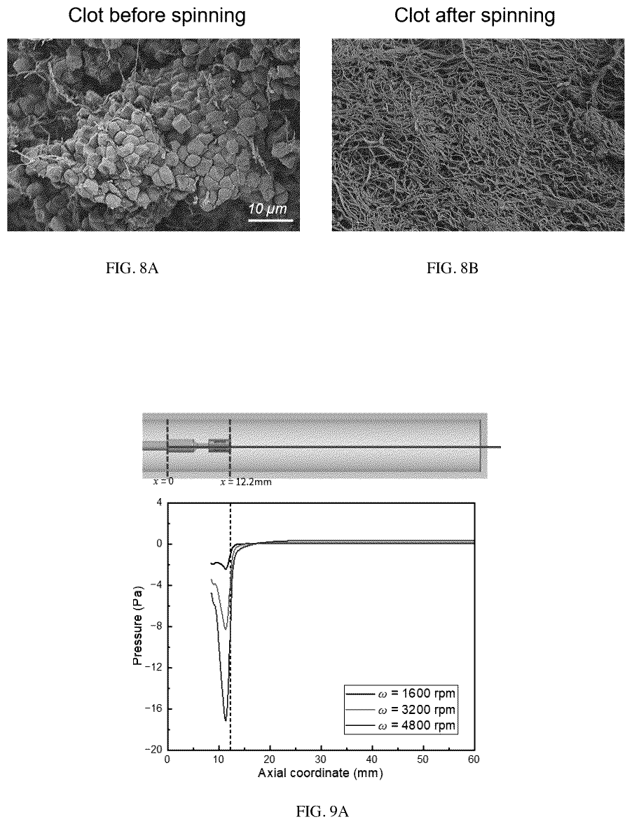

FIGS. 6A-6C show an example of the changes in a blood clot (created by pig blood in this example) during spinning the spinner tip of a thrombectomy device from zero to three minutes (0-3 min). As can be clearly seen, the size of the clot significantly reduces, and the clot color turns from red to white, as shown in FIGS. 6B and 6C. This is due to the shear force created by the spin-suction that effectively spins out all red blood cells (RBCs) from the original clot. Since a blood clot is mainly composed of RBCs trapped in fibrin fiber networks, e.g., as shown in FIG. 7, removing RBCs leaves only fibrin fiber network that is less than 10% of the initial clot volume. For example, FIGS. 8A and 8B include SEM images of a clot before and after spinning, respectively, further showing that the original clot is RBC rich and the clot after spin is highly densified fibrin fiber network.

Instead of having a sharp blade that rotates under high rpms (e.g., 150,000 to 200,000) for maceration, the spinner tip may be flexible or semi-rigid and operate under relatively low rpms (e.g., between about 4000 to 50,000) for the separation of red blood cells from fibrin network. The hole and cut features of the spinner may enhance suction to firmly compress the clot against the spinner tip distal face to ensure maximum shearing of the clot.

FIGS. 9A-9C show examples of the performance of various spinner tips under different conditions from simulations, e.g., involving positioning a spinner device within a tube, e.g., a three-millimeter tube, used to simulate operation of the spinner tip within a blood vessel. For example, FIG. 9A shows localized suction that may be generated when a spinner tip is rotated at different speeds. The localized pressure drop represents the magnitude of compression force generated, which can be tuned by varying spinning speed, thereby indicating that the clot dissolution efficiency is adjustable and may be improved with a higher spinning speed. FIG. 9B shows examples of centerline pressure profile of a spinner device with 0.87 normalized blade length (blade length, L normalized against inner radius, r) at spinning speed of 10k, 20k, 30k, and 40k rpm. FIG. 9C compares various blade lengths at 40k rpm spinning speed. The simulation was conducted for the optimization of spinner suction capability as demonstrated in FIG. 9C against normalized blade length. Optimized suction may be achieved when the normalized blade length is 0.87 and is chosen as the geometry design of the spinner tip. FIGS. 10(1) to 10(10) show additional examples of pressure distribution at the centerline of a spinner device (shown in FIG. 10) with different blade sizes during spinning.

Optionally, as shown in FIG. 4D, a drug delivery member 49 may be provided within the cavity 48 of the spinner tip 40. For example, a cylindrical body preloaded with one or more desired agents may be inserted into the spinner tip 40 immediately before a procedure or may be provided within the spinner tip 40 at the time of manufacturing. Alternatively, one or more agents may be loaded into the drug delivery member 49 immediately before the procedure and inserted into the cavity 48 to deliver the agent(s) during the procedure. For example, as described elsewhere, the agent(s) may be released as the spinner tip 40 is rotated and, optionally, the release speed of the agent(s) may be tuned to the speed of rotation of the spinner tip 40. In another option, fluid, e.g., saline and the like, may be introduced through the lumen 26 of the catheter 20 to facilitate releasing the agent(s) into the blood vessel from the spinner tip 40, if desired.

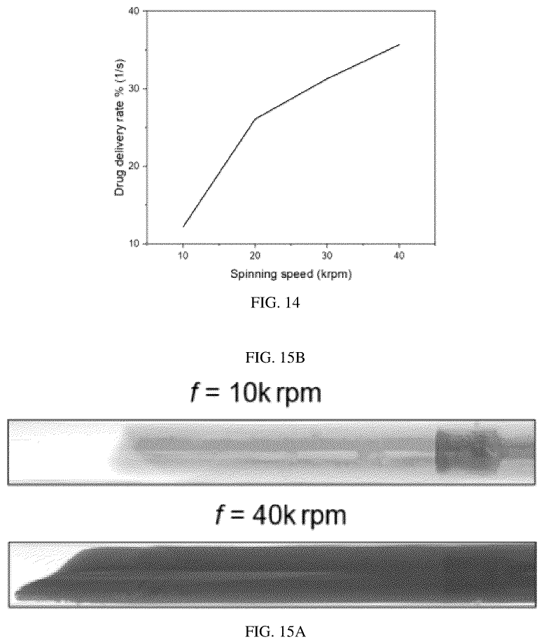

Optionally, with additional reference to FIG. 3, the controller 62 coupled to the motor 60 may be used to adjust a rotation speed of the shaft 28, e.g., to control a release rate of the one or more agents carried by a drug delivery member, e.g., one or more of clot-dissolving agents, thinning agents, anti-inflammatory agents, dyes, contrast, and/or other therapeutic and/or diagnostic agents. For example, FIG. 14 shows experimental results of controlled drug release (such as releasing the clot-dissolving medicine tPA (tissue plasminogen activator), the first treatment for acute ischemic stroke) using a spinner tip, showing the drug release rate under various spinning speeds. Under relative low spinning speeds, e.g., around ten thousand rpm, the drug release rate (expressed as a percentage of drug stored within the spinner tip that gets released per second) is lower than the release rate when the spinner tip is rotated at high spinning speeds, e.g., around forty thousand rpm. As represented by FIGS. 15A and 15B, the released dye color intensity demonstrates the different release rates, e.g., with the greater intensity shown in FIG. 15A demonstrating a faster release rate at forty thousand rpm, and the lighter intensity shown in FIG. 15B demonstrating a slower release rate. Thus, an operator may manually adjust the speed to control the release rate, and/or the controller 62 may be configured to automatically adjust the speed to provide a predetermined release rate.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

We claim:

1. A thrombectomy system, comprising:
an elongated shaft comprising a proximal end configured to be coupled to a controller to spin the elongated shaft, a distal end, and a longitudinal axis extending therebetween, the shaft configured to rotate about the axis; and
a spinner tip on the distal end, the spinner tip comprising a hollow body having a distal opening, one or more external projections extending from a lateral side of the hollow body, and one or more openings into the hollow body along the lateral side of the hollow body;
wherein the spinner tip is configured to rotate with the elongated shaft to separate fibrin from red blood cells to reduce clot size.

2. The system of claim 1, further comprising a catheter having a lumen, wherein the elongated shaft and spinner tip are configured to extend within the catheter so that the spinner tip is positioned adjacent to a distal end opening into the lumen of the catheter.

3. The system of claim 2, further comprising a stop configured to prevent the spinner tip from extending beyond the distal end opening of the catheter.

4. The system of claim 2, further comprising a proximal suction port configured to connect the lumen to a source or negative pressure.

5. The system of claim 2, wherein the elongate shaft and spinner tip are integrated with the catheter.

6. The system of claim 1, wherein the one or more external projections comprises one or more blades on the hollow body.

7. The system of claim 1, wherein the one or more external projections extend longitudinally and/or helically down the hollow body.

8. The system of claim 1, wherein the one or more openings extend longitudinally along the hollow body between the cavity and an outer surface of the annular body.

9. The system of claim 1, wherein the spinner tip is formed of a metal or polymeric material.

10. The system of claim 1, wherein the spinner tip is formed of a flexible material.

11. The system of claim 1, wherein the distal face of the spinner tip is substantially flat.

12. The system of claim 1, wherein the spinner tip is configured to prevent maceration of the clot when rotated.

13. The system of claim 1, further comprising a motor configured to couple to the proximal end of the shaft to rotate the shaft about the axis and a controller coupled to the motor end for controlling rotation of the shaft.

14. The system of claim 13, wherein the controller is configured to rotate the shaft at least 1,000 rpm.

15. The system of claim 1, further comprising a sleeve extending over the elongate shaft.

16. A thrombectomy system, comprising:
an elongated shaft comprising a proximal end configured to be coupled to a controller to spin the elongated shaft, a distal end, and a longitudinal axis extending therebetween, the shaft configured to rotate about the axis; and
a spinner tip on the distal end, the spinner tip comprising a hollow body forming a cavity having a distal face with a distal opening, one or more external projections extending from a lateral side of the hollow body, and one or more openings into the cavity of the along the lateral side of the hollow body;
wherein the spinner tip is configured to rotate with the elongated shaft to generate a localized suction to apply compression and shear forces against a clot contacting the distal face to separate fibrin from red blood cells to reduce clot size.

17. The system of claim 16, further comprising a catheter having a lumen, wherein the elongated shaft and spinner tip are configured to extend within the catheter so that the spinner tip is positioned adjacent to a distal end opening into the lumen of the catheter.

18. The system of claim 17, further comprising a stop configured to prevent the spinner tip from extending beyond the distal end opening of the catheter.

19. The system of claim 17, further comprising a proximal suction port configured to connect the lumen to a source of negative pressure.

20. The system of claim 16, wherein the one or more openings extend longitudinally along the hollow body between the cavity and an outer surface of the annular body.

21. The system of claim 16, wherein the spinner tip is formed of a metal or polymeric material.

22. The system of claim 16, wherein the spinner tip is formed of a flexible material.

23. The system of claim 16, wherein the distal face of the spinner tip is substantially flat.

24. The system of claim 16, wherein the spinner tip is configured to prevent maceration of the clot when rotated.

25. The system of claim 16, further comprising a motor configured to couple to the proximal end of the shaft to rotate the shaft about the axis and a controller coupled to the motor end for controlling rotation of the shaft.

26. The system of claim 25, wherein the controller is configured to rotate the shaft between 1,000 and 50,000 rpm.

27. The system of claim 25, wherein the controller is configured to rotate the shaft at least 1,000 rpm.

28. The system of claim 16, further comprising a sleeve extending over the elongate shaft.

29. A thrombectomy system, comprising:
an elongated shaft comprising a proximal end configured to be coupled to a controller to spin the elongated shaft, a distal end, and a longitudinal axis extending therebetween, the shaft configured to rotate about the axis;
a spinner tip on the distal end, the spinner tip comprising a hollow body forming a cavity having a distal face with a distal opening, one or more external projections extending from a lateral side of the hollow body, and one or more openings into the cavity of the along the lateral side of the hollow body;
a catheter having a lumen, wherein the elongated shaft and spinner tip are configured to extend within the catheter lumen; and
configured to prevent the spinner tip from extending beyond a distal end opening of the catheter;
wherein the spinner tip is configured to rotate with the elongated shaft to separate fibrin from red blood cells to reduce clot size.

30. A method of performing a thrombectomy, the method comprising:
contacting distal face of a spinner tip against a clot;
rotating the spinner tip, wherein the spinner tip comprises a hollow body forming a cavity having a distal opening, one or more external projections extending from a lateral side of the hollow body, and one or more openings into the cavity along the lateral side of the hollow body;
applying a shear force between the clot and the distal face to separate fibrin from red blood cells within the clot and reduce clot size, wherein the clot is driven against the distal face by the application of suction while rotating the spinner tip.

31. The method of claim 30, wherein contacting comprises positioning a distal end of a catheter adjacent to the clot wherein the spinner tip is within the catheter and adjacent to the distal end of the catheter.

32. The method of claim 31, further comprising applying suction through the catheter.

33. The method of claim 31, further comprising preventing the spinner tip from extending distally beyond the distal end of the catheter.

34. The method of claim 30, further comprising inserting the spinner tip through a catheter positioned within a blood vessel, wherein a distal end of the catheter is adjacent to the clot.

35. The method of claim 30, wherein rotating the spinner tip comprises rotating at between 1,000 and 50,000 rpm.

36. The method of claim 30, wherein rotating comprises rotating a shaft to which the spinner tip is coupled.

37. The method of claim 30, wherein applying the suction comprises generating a localized suction by rotating the spinner tip.

38. The method of claim 37, wherein generating the localized suction comprises generating a pressure drop of greater than 500 Pascals (0.5 kPa).

39. The method of claim 37, wherein generating the localized suction comprises rotating the spinner tip for less than 2 minutes.
